(19) United States
(12) Patent Application Publication
(10) Pub. No.: US 2017/0021113 A1
(43) Pub. Date: Jan. 26, 2017

(54) IMPROVED INSTRUMENT, ARTICLE AND METHOD FOR MINIMIZING PAIN

(71) Applicant: Bing Innovations LLC, Boca Raton, FL (US)

(72) Inventors: Steven G. GOLDBERG, Boca Raton, FL (US); Mark LORBERBAUM, Boca Raton, FL (US); Gary A. AYOTTE, Newburyport, MA (US)

(73) Assignee: Bing Innovations LLC, Boca Raton, FL (US)

(21) Appl. No.: 15/039,898

(22) PCT Filed: Nov. 26, 2014
(86) PCT No.: PCT/US14/67587

§ 371 Date: May 27, 2016

Related U.S. Application Data
(60) Provisional application No. 61/909,544, filed on Nov. 27, 2013.

(51) Int. Cl. A61M 5/42

(52) U.S. Cl. CPC A61M 5/422; A61M 2205/273; A61M 2205/587; A61M 2205/8243

(57) ABSTRACT

An instrument is described for minimizing pain during administration by injection of a liquid, such as, an anesthetic. The instrument includes a main body having a rod mounted freely for vibration and a single use tip removably mounted on the forward end of the main body by a first type of motion and removable by a second and different type of motion. The tip is held on the instrument by an elastic overmold. A vibration unit imparts vibrations to the tip. Demounting of the single use tip will rupture the overmold and render the single use unfit for further use. A method of operating the instrument is described and a single use tip is described.

### RELATED CASES

The invention is an improvement to the instrument, article and method described in US Published Patent Application 20120029422 published Feb. 2, 2012 the text and drawings of which are here incorporated in their entirety.

### SUMMARY OF THE INVENTION

An improved instrument, article and method are provided for minimizing pain during administration by injection of a liquid, such as, an anesthetic. The instrument has a forward end. A rod or lightpipe is mounted freely for vibration projects out of the forward end. The article, a single use tip, is composed of a tip sleeve removably mounted on the forward end of the instrument and a tip member removably mounted on the projecting rod or lightpipe to vibrate a preselected injection site on a human or animal. The tip sleeve and tip member are covered by an elastic overmold that enables the tip member to vibrate freely with respect to the tip sleeve and light from the lightpipe to illuminate the injection site. The overmold of the single use tip is torn during removal of the single use tip from the instrument by a novel structure for the front end and the tip member.

### BRIEF DESCRIPTION OF THE DRAWINGS

The above-mentioned features and objects of the present invention will be discussed in terms of the attached figures wherein like reference numerals denote like elements and in which:

FIG. 1 a perspective view of the instrument with a disposable tip mounted thereon;

FIG. 2 is an exploded drawing of FIG. 1 showing an embodiment of the apparatus of the present invention with a disposable tip;

FIG. 3 shows a perspective view showing the disposable tip member or sleeve;

FIG. 4 is a perspective view showing the handle tip member or sleeve;

FIGS. 5 to 10B show in various rotated views improvements to the handle tip member or sleeve;

FIGS. 11 to 15 show in various views improvements to the disposable tip;

FIGS. 16 to 20 show in various views in detail improvements to the disposable tip sleeve of the disposable tip;

FIGS. 21 to 24 show in various views a disposable tip elastomer or overmold;

FIGS. 25 to 27 show in various views a disposable tip core.

### DETAILED DESCRIPTION

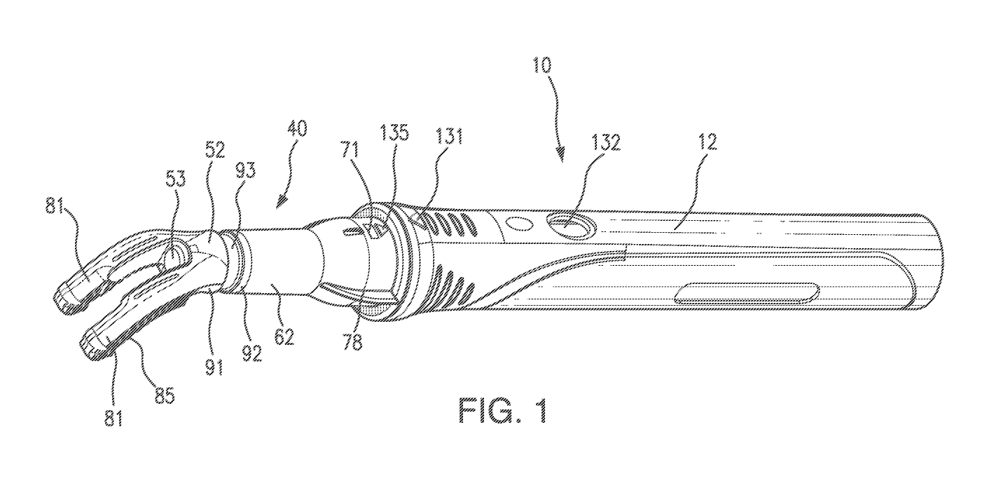

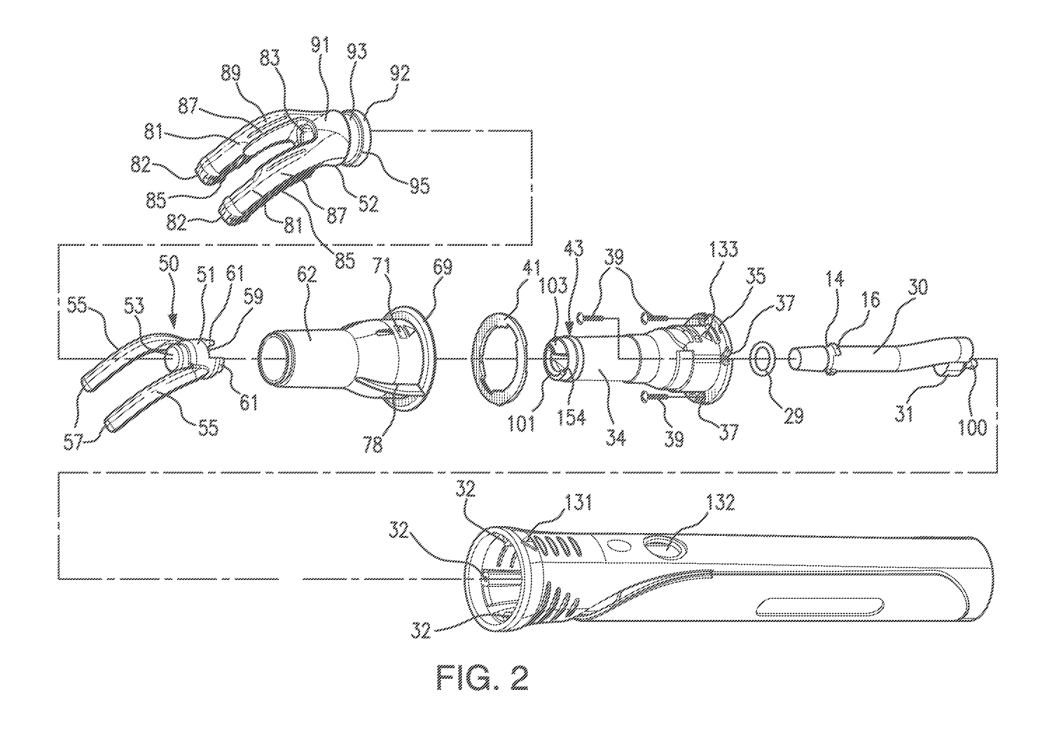

Referring to the drawings, an embodiment of the retractor vibrator is shown generally in FIG. 1, designated as 10 for performing the required. An exploded view is shown in FIG. 2, in particular, retractor vibrator 10 is a handheld apparatus comprising a main body or handle 12 in the shape of a tube having a battery cover and an end closure that is threaded to screw onto the open end of handle 12 in a complementary fit. This is as described in WO/2010/111611, hereby incorporated by reference in its entirety. Further as described in WO/2010/111611 handle 12 is composed of hard plastic and partially receives a chassis, the uncovered portion of which is covered by a battery cover. Rechargeable batteries, not shown in FIG. 1, are located in a battery compartment of the chassis. An induction coil is mounted on the end of chassis and positioned in the battery cover to couple to a charge device in a conventional manner. A motor 24, not shown, drives eccentrically a cam and is housed in the handle 12. The cam includes a ball socket whose axis is offset from the motor drive axis by from about 0.010 inches to about 0.025 inches, with an offset of about 0.020 inches being preferred. The vibration induced by the cam and its follower 100 fixed to rod 30 via a fitting at its rear end is transmitted via polycarbonate light rod 30 to polycarbonate tip 50 firmly engaged demountably with rod 30 at its forward end. The vibrations produced at the free ends of the two bifurcated legs 42 of tip 50 will have amplitude of about 0.5 mm (0.020 inches).

During vibration, the light rod 30 is free from all other structure except the cam 26 at its rear end and tip 50 at its forward end so that it can vibrate freely by pivoting about resilient ring 29 (rubber or plastic O-ring) mounted in a groove 14 formed in rod 30 spaced from the forward end of rod 30. The rod 30 has projections 16 on the rear side of the groove 14 to assist in holding the O-ring 29 in correct position. The forward end of the rod 30 firmly engaged with tip 50. Also as in the structure described in WO/2010/111611, a PCB board containing electrical and electronic circuitry is mounted on the chassis (not shown). The battery (not shown) is connected via the circuitry to control the motor in the manner described in WO/2010/111611 here incorporated by reference. When the motor is driven, vibration produced by the cam is coupled to polycarbonate light rod 30 via a coupling fitting and cam follower 100. Alight, preferably an LED, is positioned at the rear end of the rod 30 and powered by the battery and circuitry, as described in WO/2010/111611.

The forward end of the handle 12 is provided with 4 bolt mountings 32, see FIG. 2. A handle sleeve 34 has a flange 35 at its rear end provided with 4 bolt openings 37 to receive bolts 39 to fix the handle sleeve 34 in position to the handle 12. A cover annulus 41 is fitted into the handle 12 to close the opening and cover the bolts 39. The light rod 30 passes through the handle sleeve 34 and projects outwardly from it. The O-ring 29 resiliently engages the inner surface of the handle sleeve 34 near its forward end. The front end of the handle sleeve 34 has a low flange 43 set back from the open front end.

The handle sleeve 34 receives a single use disposable tip 40 comprised of three components, namely, a forward plastic tip 50 that is firmly but detachably mounted on the end of light rod 30, a tip sleeve 62 for detachably and rigidly being mounted on handle sleeve 34 and a resilient (rubber or plastic) overmold 52 that covers the plastic tip 50 and holds tip 50 and tip sleeve 62 together in a spaced relationship that enables tip 50 to vibrate freely relative to tip sleeve 62.

The components of the disposable tip 40 consist of plastic tip 50 consisting of a cup 51 with its closed end defining a lens 53. Two curved protrusions or legs 55 extend from the sides of the cup 51 bracketing the lens 53, extending forwardly and terminating in free ends 57 that are rounded. On its rear facing side, the cup 51 has a cutout 59 and two tongues, tabs or positive ribs 61 that extending rearward, one on each side of the cutout 59. Diametrically opposite cutout 59 is a small cutout 63. The rod 30 enters into the cup 51 and firmly and rigidly engages the cup 51 and the forward end of the rod 30 is juxtaposed closely to the lens 53.

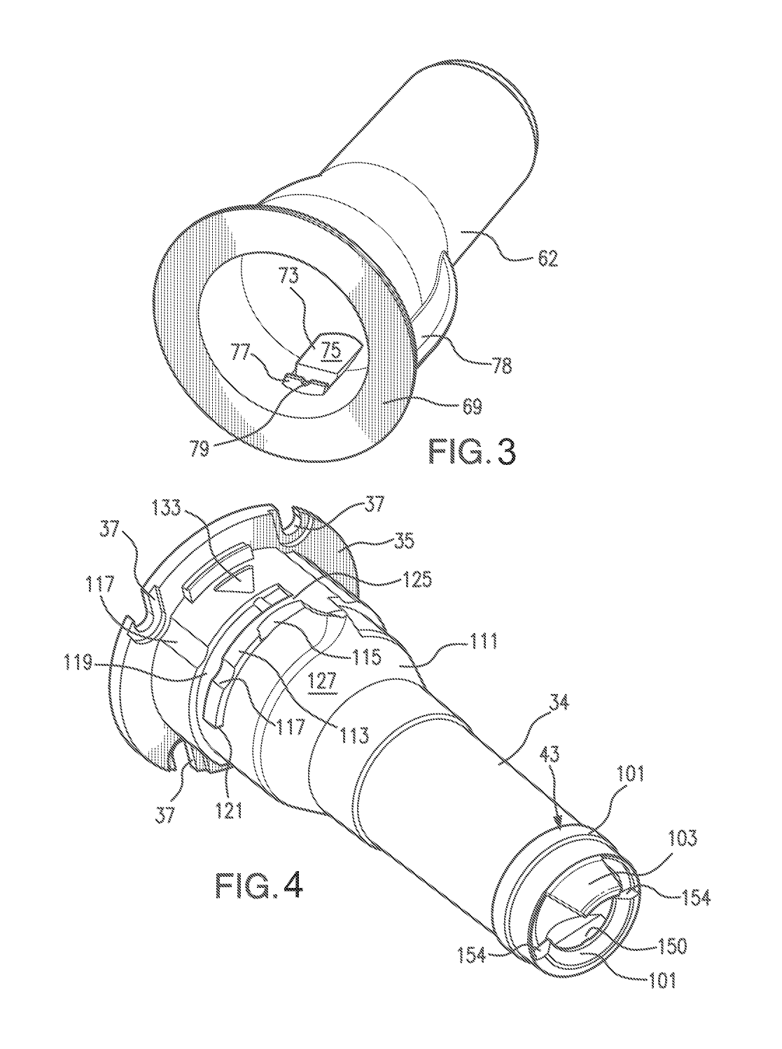

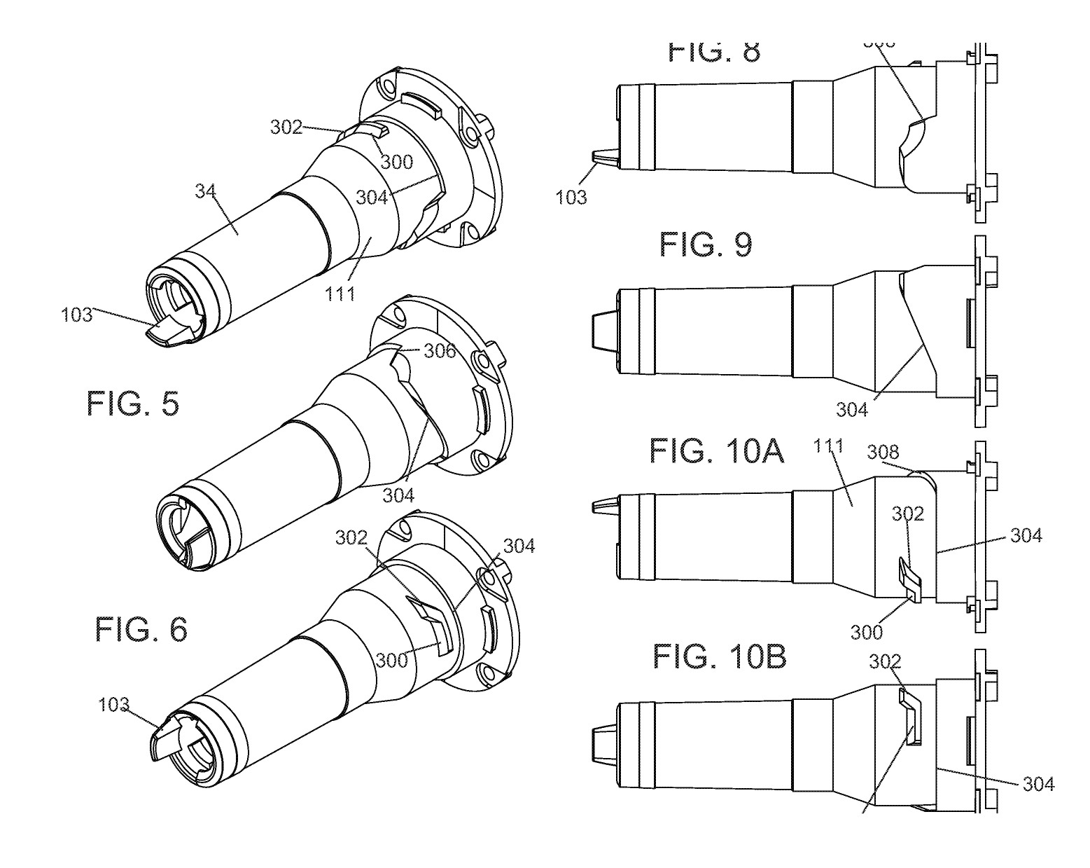

The tip sleeve 62, see FIG. 3, consists of a tube having at its forward end a ridge 65 and behind it a groove 67. At its rear end, tip sleeve 62 increases in diameter conically and terminates in a flange 69. Adjacent its rear end, a rectangular through opening 71 is formed in the shell of the tip sleeve 62. On the forward side of the opening 71 the inside of the tip sleeve 62 is thickened at 73 to form a flat shelf 75 generally the circumferential width of the opening 71. On the rear side of the opening 71 the inside of the tip sleeve is thickened to form an inwardly extending plate or rib 77 also generally the circumferential width of the opening 71. Plate 77 has a v-notch 79 formed at its inmost extent. Reinforcing ribs 78 is on the exterior of the tip sleeve 62 extending from the conical section to the flange 69.

Overmold 52 is hollow and the forward portion has two longitudinally extending arms 81 that fit over the longitudinally extending legs 55. The free ends of the arms terminate in an enlarged end bulbous portion 82. The overmold 52 is composed of rubber or a thermoplastic elastomer having a durometer of preferably about 40 A but may vary from about 30 A to about 50 A. Portion 91 of the overmold fits over the cup 51 and has a hole 83 that fits around the lens 53 to let the light shine through. The two arms 81 have a series of transversely extending, longitudinally spaced grooves 85 on the underside, defined by projecting filaments, to trap saliva so reuse of the disposable tip will not be attempted. The two arms 81 of the overmold 52 can be smooth or have many filaments in the form of ribs as shown, or an array of projecting filaments of any shape, projecting off of the surface of the overmold arms 81, providing a greater stimulation to the tissue while in use. An intermediate portion 87 is enlarged transversely on both arms 81 of the overmold 52 to collectively form a retractor surface 89 to help press away or retract tissue. Rear portion 92 of the overmold 52 has a reduced thinned section 93 that at the rear is integral with a terminal section 95 complementary in configuration to the forward end of the tip sleeve 62 to fit over the ridge 65 and behind it in the groove 67 to anchor the overmold 52 to the tip sleeve 62 and holds it spaced from the tip 50.

The handle sleeve 34, as shown in FIG. 4 is a longitudinally extending tube and has at its forward end, a circumferential radially inward flange 101 that terminates spaced from either side of a tapered protrusion or tooth 103 extending axially or longitudinally forward level with the forward end of the sleeve 34, to define recesses 154. The tooth 103 extends slightly into the sleeve 34 forming a rear shoulder 152. Circumferential flange 101 formed at the forward end of the handle sleeve 34 serves as a constraint on the forward side to hold the O-ring 29. O-Ring 29 also passes behind and bears on shoulder 152 of tooth 103 and thus, is constrained on the forward side also by the tooth 103. These elements together hold the light rod 30 on the forward side in position within the forward end of handle sleeve 34. On the rear side, projection 16 on the light rod is constrained by arcuate radially inwardly projections 150. Also recesses 154 are formed on either side of the tooth 103 between the terminating ends of the circumferential flange 101 and the tooth 103. Recesses 154 are about 1-2 mm deep. As noted, only the O-ring 29 engages the inner surrounding surface of handle tip 34. The rod 30 with the O-ring 29 acting as a pivot is otherwise completely free to vibrate.

The handle sleeve 34 at its rear end is of larger diameter with a transition conical section 111 between the front end and rear end. The rear end terminates in flange 35 having bolt opening 37. Spaced forward of the flange 35 is a surface portion 127 of the conical section 111 that is of fixed diameter. An arcuate ridge 113 is formed that extends circumferentially from a point 125 of larger diameter than portion 127 circumferentially for about 60 to 90 degrees and terminates at point 121. Ridge 113 is narrowed by cutout 115 on its right side as viewed in FIG. 4 and has a dip 117 to enable a bolt to pass through to a bolt opening 37. The ridge 113 defines a groove 119 on its rear side, due to surface 127 having a fixed diameter and a low rib 123 extends longitudinally in groove 119 to the rear of cutout 115.

Each of tip sleeve 62, handle sleeve 34 and handle 12 have indicia to show how the parts line up. Handle 12 has an arrowhead 131 pointing forward on its front end aligned with the control button 132 to turn the power on and off. Handle sleeve 34 has an arrowhead 133 pointing forward just behind the flange 35. The disposable tip 40 has an arrowhead 135 on tip sleeve 62 pointing rearward so that when the disposable tip 40 is pushed longitudinally onto the handle sleeve, the tooth 103 will be inserted or protrude into the cutout 59 of the plastic tip 50, but with clearance from both the plastic tip 50 and light rod 30 so that vibration of tip 50 by light rod 30 is not interfered with in any way. Also, the rear ends of positive ribs 61 will be received and positioned in recesses 154 in the handle sleeve 34, but with clearance from the handle sleeve 34 and tooth 103 so that tip 50 vibration is not interfered with in any way. The positive ribs 61 of tip 50 and recesses 154 of handle sleeve 34 are complementary and the clearance between them is at least about 0.5 mm.

As the disposable tip 40 is being pushed onto the handle sleeve 34 in a linear longitudinal direction, the thickened area at 73 that forms a flat shelf 75 causes the inwardly extending plate 77 to ride over the ridge 113 at cutout 115 and go behind into the groove 119, whereupon the flat shelf 75 drops down onto the surface 127, the rectangular through opening 71 straddles the ridge 113 at the cutout 115 and the plate 77 drops onto the rib 123 with the v-notch 93 engaging the rib 123. At this point, the rod 30 is received in and positioned in the cup 51 of the tip 50 of the disposable tip 40, which is latched onto the handle sleeve 34 and cannot be pulled off axially. The tooth 103 at the forward end of handle sleeve 34 is received in and positioned in the cutout 59 with suitable clearance all-around of at least about 0.5 mm so that there is not any interference respecting vibration. Also, the rear ends of the positive ribs 61 on either side of the cutout 59 are received in the recesses 154 to a sufficient depth to enable back-up engagement with the handle sleeve 34 in the event there is any relative rotary motion with respect to the handle sleeve 34 and the tip 50. Both the tooth 103 and the rear ends of the positive ribs 61 serve to hold the handle sleeve 34 and the tip 50 from relatively rotating. The tooth 103 and the positive ribs 61 will, at this time, be juxtaposed in the same radial cylinder coaxial with the longitudinal axis of the instrument, the handle sleeve 34 and the cutout and the tabs 61 of cup 50.

To remove the disposable tip 40 from the handle sleeve 34, it is necessary for the tip sleeve 62 to be rotated relative to the handle sleeve 34 from adjacent point 125 toward the terminal point 121 until the plate 77 clears this point 121, at which time, the disposable tip 40 can be removed by longitudinally puffing forward. However, during the relative rotary motion between the tip sleeve 62 and handle sleeve 34, the plastic tip 50 will quickly engage the tooth 103, either by the side was of the cutout 59 or the tabs or positive ribs 61 or both and the tip 50 will then be held stationary and prevented from rotating. The tip sleeve 62 will still be rotatable relative to the handle sleeve 34 so it can be removed, and in this process as the tip sleeve 62 is rotated, a torsion force will develop and be imparted to the overmold 52 and the elastic limit of the overmold 52 at the thinned section 93 will quickly be exceeded and the overmold will experience a severe tearing or shredding of the thinned section 93 of overmold 52 over about 60 to 90 degrees or more destroying effectively its utility and/or its ability to be reused. The rear end of the positive ribs 61 of tip 50 being in the recesses 154 is a back-up in case of any failure of the tooth 103 to hold the tip 50 stationary during demounting of the disposable tip 40 from the sleeve 34. The rear ends of the positive ribs 61 being positioned in recesses 154 will also prevent any rotation of tip 50 relative to handle sleeve 34 during the demounting of disposable tip 40 and thereby insure the tearing or shredding of the thinned section 93 of overmold 52.

As described, the fitting member 31 attached to the end of the light rod 30 includes a projecting stub with ball end 100 as a cam follower for contacting and following the cam 26 to impart the vibratory motion of the cam 26 to the light rod 30. The end of the light rod 30 is in alignment with the LED 36 as previously described. Although continuous pulsing can be used, it has been discovered that a pulsed vibration sequence is more effective. A pulsed sequence of about one second on and then about a tenth of a second off, is sufficient to pulse the vibration without allowing the vibration of the tip to ever drop down to zero. In this manner, the device or instrument pulses about every second, re-stimulating the nerves in the area, and apparently the brain never gets used to it, so the vibrations remain effective. To this end the electronics controlling the pulsing is modified to include the necessary means for pulsing and obtaining the desired on/off duty cycle as noted. The motor is energized using 110 volt AC (50-300 Hz frequency). The frequency can vary from about 50 Hz to about 300 Hz with about 140 Hz to 160 Hz being preferred. The vibration at the free ends of the prongs or forks 42 can vary from about 0.1 mm to about 1.0 mm with the preferred range being 0.65 mm to 1.0 mm. The duty cycle is about 1.05 seconds with on pulsing of about 1 sec. and off of about 0.05 sec. The vibration amplitude may vary from about 0.1 mm to about 1.0 mm, with the preferred range being from about 0.65 mm to about 1.0 mm.

The instrument exhibits enhanced amplitude and percusses the tissue contacted, causing a deeper penetration of the vibratory stimulus into the oral mucosa of a dental patient, or into the skin of a medical patient, thereby stimulating the sensory nerve receptors, which are located deep within the tissue, creating action potentials which send sensory information to the brain via myelinated A-beta afferent nerve fiber axons.

Also, vibration is sent out in a full 360 degrees around the disposable tip 40, mainly, from three places off of the tip, namely, from each free end of the curved downwardly arms, as well as, from the paddle or retractor portion 87. Also the joint with tip sleeve 62 at times will stimulate the lip or cheek or bone when resting against it. The enhanced vibration is not limited to just the area at the ends of the curved downward arms. Alternative tip free end shapes are shown in WO/2010/111611, incorporated herein by reference.

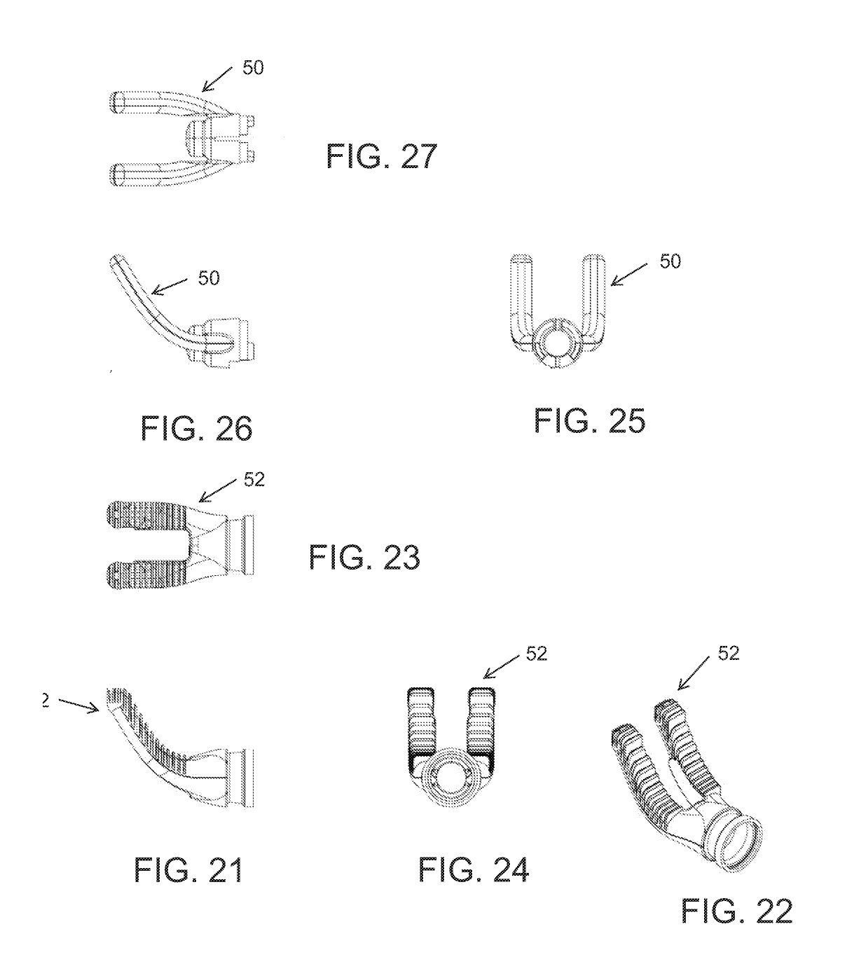

The improvement to the above structure involves the handle forward tip member or sleeve 34 and the disposable tip member of sleeve 62. The same reference numbers are given to the same parts in FIGS. 8 to 27. The handle forward tip member or sleeve 34 is constructed similarly to the prior design at it end that engages the disposable tip core. At its end mounted to the main body 12, the outer surface is differently profiled. The ramp 111 is present and a projecting rib 300 is formed circumferentially extending for a short distance terminating at one end 302 in a short section angled forward toward the tip core 50. Rib 300 is positioned on sleeve 34 diametrically opposite the tooth 103, in the opposite position to where it was in the prior instrument. A circumferentially extending space is defined behind the rib 300 bordered axially spaced by a shoulder 304 that also runs circumferentially. As shown in the clockwise direction in the drawing, the shoulder 304 defines a stop 306 whereas in the counterclockwise direction it defines a curving 308 forward toward the tip core 50.

The disposable tip member or sleeve 62 is modified to define an elongated circumferentially extending slot 320 that extends circumferentially more than the rib 300. The slot 320 is on the underside opposite the indicia printed on the top side of tip 40. A depending rib 322 is circumferentially defined inside the sleeve 62 that borders from one end to the other end of the slot 320 adjacent the end of sleeve 62 that is remote form the tip core 50. A shelf 324 is formed leading to the slot 320.

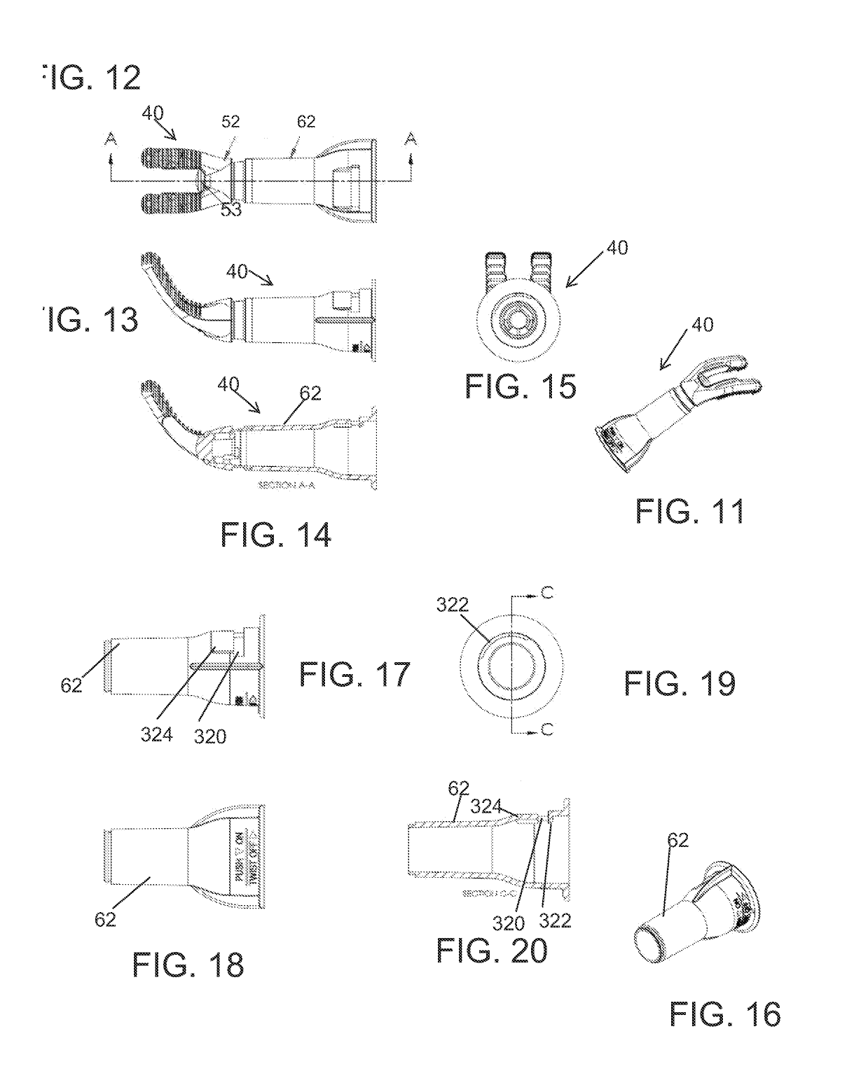

When the disposable tip 40 is mounted on the handle forward tip member or sleeve 34, the tip sleeve 62 is pressed axially straight on the handle tip member. The slot 320 partially aligning with the rib 322 will pass over the rib 300 so that the depending rib 322 is positioned in the circumferential space against the shoulder 304. Any attempt to rotate the tip sleeve 62 to the left as shown in FIG. 11 will result in the end of rib 322 striking the stop 306. When the tip sleeve 62 is to be removed, it will be rotated to the right as shown in FIG. 11 whereupon the other end of the rib 322 will follow the shoulder 304 and due to the curving 308 will be pushed off the sleeve 34. To this end the angled section at end 302 enables clearance for the rib 322 so the disposable tip 40 can be pulled off the handle forward tip member or sleeve 34. The rotation of the disposable tip will cause tearing of the overmold to render the disposable tip 40 unfit for further use.

All references cited herein are expressly incorporated by reference in their entirety. It will be appreciated by persons skilled in the art that the present invention is not limited to what has been particularly shown and described herein above. In addition, unless mention was made above to the contrary, it should be noted that all of the accompanying drawings are not to scale. There are many different features to the present invention and it is contemplated that these features may be used together or separately. Thus, the invention should not be limited to any particular combination of features or to a particular application of the invention. Further, it should be understood that variations and modifications within the spirit and scope of the invention might occur to those skilled in the art to which the invention pertains. Accordingly, all expedient modifications readily attainable by one versed in the art from the disclosure set forth herein that are within the scope and spirit of the present invention are to be included as further embodiments of the present invention.

What is claimed is:

1. In an instrument for minimizing pain during administration by injection of a liquid, such as, an anesthetic comprising a main body having a forward end, a rod mounted in said body freely for vibration and projecting out of said forward end of said body, a single use tip composed of a tip sleeve removably mounted on the forward end of said main body by a first type of motion and removable from said main body by initiating a second and different type of motion, and a tip member having a shape to vibrate a defined preselected injection site on a human or animal, said tip member being removably and rigidly mountable on the rod, said tip member and tip sleeve being held together by an elastic overmold but in a spaced apart juxtaposition that enables said tip member to vibrate freely relative to said tip sleeve, a vibration unit mounted in the main body and coupled to said rod to impart vibrations via said rod to said tip member, wherein said main body includes a forwardly projecting element and said tip member defines a configuration to coact with said projecting element such that the removable demounting of the single use tip will rupture the overmold and render the single use unfit for further use the improvement comprising the forward end of the main body defining a first rib with an angled end and an axially spaced shoulder having a curving section and the tip sleeve defining an elongated circumferential slot with a second depending rib for passing over the first rib and lying in the space between the first rib and the shoulder.

2. An instrument according to claim 1 wherein the configuration of the tip member includes both a cutout and tabs projecting toward the main body, and the projecting element is a tooth.

3. An instrument according to claim 1 wherein the instrument is elongated longitudinally and the first type of motion is linear and the second type of motion is rotary.

4. An instrument according to claim 1 wherein the tip sleeve is slid longitudinally onto the front of the instrument and rotated relative to the front of the instrument to remove.

5. An instrument according to claim 1 further including mutually coacting elements on the tip sleeve and the main body of the instrument that latch the tip sleeve to the main body.

6. An instrument according to claim 5 wherein the mutually coacting elements include a rib and a v-notch plate.

7. An instrument according to claim 1 further comprising a controller for controlling the vibration unit for an on-off duty cycle,

8. An instrument according to claim 7 wherein the duty cycle is about 1 second on and about 0.05 seconds off.

9. An instrument according to claim 1 wherein the overmold covers the tip member and wherein at least one groove is defined in the overmold for collection of saliva.

10. An instrument according to claim 1 wherein the overmold covers the tip member, and wherein the portion of overmold surface for contacting the defined preselected injection site is smooth, has projecting filaments or is formed with a bulb.

11. An instrument according to claim 1 wherein the rod is a lightpipe and a light shines through the lightpipe and exits through the tip member to illuminate an injection site.

12. A method for injecting a liquid at a preselected site on tissue of a human or animal comprising the steps of providing an instrument including a main body defining a first rib with an angled end and an axially spaced shoulder having a curving section and a disposable tip having a tip sleeve defining an elongated circumferential slot with a second depending rib for passing over the first rib and lying in the space between the first rib and the shoulder, whereby said main body and said disposable tip are detachably mounted together, said disposable tip including a mounting part for mounting on the main body and a vibrating tip held together by a resilient overmold, creating vibrations in said main body, transmitting the created vibrations to the vibrating tip, applying the vibrating tip to tissue at a preselected injection site to vibrate the tissue, simultaneously injecting by a needle or like instrument a liquid into the preselected injection site, and tearing the overmold to render the disposable tip unfit for further use.

13. The method of claim 12 further including the step of lighting the injection site via the disposable tip.

14. The method of claim 12 further including the step of simultaneously pulsing said vibration.

15. The method of claim 12 further includes continuing to vibrate the injection site after injection to dissipate any tissue distension and simultaneously illuminating the injection site

16. The method of claim 15 wherein continued vibration includes applying greater pressure to the tissue to sound bone.

17. The method according to claim 14 wherein the pulsing duty cycle is about 1 second on and about 0.05 seconds off.

18. The method of claim 14 wherein the amplitude of vibration of the vibrating tip is from about 0.1 mm to about 1.0 mm.

19. A single use disposable tip for use with an instrument that includes a vibration unit and a rod that is vibrated by the vibration unit, the rod projecting out of the forward end of the instrument, said tip comprising a tip sleeve for removably mounting on the forward end of instrument and a tip member for removably and rigidly mounting on the projecting rod, said tip member having a shape for vibrating a defined preselected injection site on a human or animal, said tip sleeve and tip member being covered and held together in spaced juxtaposition by an elastic overmold that enables the tip member to vibrate freely with respect to the tip sleeve, said tip sleeve defining an elongated circumferential slot with a depending rib that requires the tip sleeve to be put on a forward part of the instrument that defines a rib with an angled end and an axially spaced shoulder having a curving section with one type of motion and removed by another type of motion, and said tip member having a configuration that coacts with the instrument to remain stationary relative to the instrument when the tip sleeve is manually rotated so that the elastic overmold is torn.

20. A single use disposable tip according to claim 19 wherein the tip member comprises a cup and an integrally formed longitudinally extending forward portion having a free end, and wherein the overmold of the forward portion defines in part at least one retraction surface.

21. A single use disposable tip according to claim 20 wherein a plurality of transverse grooves are formed in the tip member.

22. A single use disposable tip according to claim 20 wherein the tip member defines a retraction area.
